(19) United States
(12) Patent Application Publication
(10) Pub. No.: US 2012/0022512 A1
(43) Pub. Date: Jan. 26, 2012

(54) DEVICE, APPARATUS, AND METHOD OF ADIPOSE TISSUE TREATMENT

(76) Inventor: Boris Vaynberg, Zikron Yaakov (IL)

(21) Appl. No.: 13/246,838

(22) Filed: Sep. 27, 2011

Related U.S. Application Data
(63) Continuation-in-part of application No. 12/665,916, filed on Aug. 10, 2011.
Application No. 12/665,916 is a continuation-in-part of application No. 12/357,564, filed on Jan. 22, 2009.
(60) Provisional application No. 61/085,424, filed on Aug. 1, 2008.
(60) Provisional application No. 61/023,194, filed on Jan. 24, 2008.

(51) Int. Cl. A61B 18/22; A61B 18/20; A61B 18/18

(52) U.S. Cl. 606/15; 606/16; 606/14; 606/33

(57) ABSTRACT

A method and apparatus for adipose tissue treatment whereby two types of electromagnetic radiation are applied to the volume of tissue to be treated, One type of the electromagnetic radiations being RF and the second type of electromagnetic radiation being visible or infrared radiation.

The present application is a continuation-in-part of the national phase application filed under 37 CFR 371 on Dec. 21, 2009 and assigned Ser. No. 12/665,916, which application is based on Patent Cooperation Treaty filing PCT/IL2009/000695, which claims priority to United States Provisional Application for Patent filed on Aug. 1, 2008 and assigned Ser. No. 61/085,424 and, the present application is a continuation-in-part of the United States patent application that was assigned Ser. No. 12/357,564, filed on Jan. 22, 2009, which application claims priority to the United States Provisional Application for patent that was filed on Jan. 24, 2008 and assigned Ser. No. 61/023,194

### TECHNICAL FIELD

The present device, apparatus, and method relate to the field of adipose tissue treatment and aesthetic body sculpturing.

### BACKGROUND

Liposuction is a popular technique for removal of fat from different sites of a subject's body. The process changes the external contours of the body and sometimes is described as body sculpturing. The fat is removed by a suction device via a cannula inserted into the appropriate site of the body. The process is painful and sometimes causes excessive bleeding.

Recently, improvements have been realized in liposuction procedures by the utilization of electro-magnetic energy or radiation such as an infrared laser radiation delivered through a fiber inserted into a cannula introduced into the treatment site. Laser radiation liquefies the adipose tissue. The liquefied tissue is either removed by suction or left in the subject body, where it gradually dissipates in a uniform way. Laser assisted liposuction is considered to be a more advanced and less invasive procedure when compared to traditional liposuction techniques.

For proper treatment, laser assisted liposuction requires application of high power ten to fifty watt laser energy or radiation. The radiation is applied in a continuous or pulse mode for relatively long periods. Sometimes more than one laser is used on the same treated tissue volume to speed up the treatment. Each of the lasers may operate in a different mode. For example, one of the lasers heats the target tissue volume, and the other one introduces laser power sufficient to destroy the adipose tissue in the same volume. This increases the cost of the equipment and prolongs the treatment session time. In addition, frequent cleaning and maintenance of the fiber tip from process debris will be required. All of the above slows down the treatment process, and in addition affects comfort and cost of procedure to the treated subject.

The present method provides an improvement over currently available techniques addressing these and other existing liposuction problems.

### Glossary

The term “mono-polar configuration” as used in the present disclosure means a configuration consisting of an active treatment electrode and a passive treatment electrode, the latter of which acts as the grounding electrode. Typically, the electrodes are different in size and can be located at a substantial distance from each other. RF induced current affects the tissue area/volume that is proximate to the active electrode.

The term “bi-polar configuration” as used in the present disclosure means that the current passes between two almost identical electrodes that are located a short distance apart from each other. The electrodes are applied to the area/volume of tissue to be treated and the propagation of the current is limited to the area between the electrodes themselves.

The term “needle” or “probe,” as used in the text of the present disclosure means a flexible or rigid light guide configured to be inserted during use into the subject tissue in order to deliver laser energy to a target volume of adipose tissue. In certain embodiments, the needle can be equipped with electrodes and configured during operation to apply RF energy to the treated tissue. The needle can also be configured to conduct a fluid to any part of the needle, and liquefied fat and the fluid from the target volume may be withdrawn. The needle may be a disposable or reusable needle.

The term “tissue” or “skin” as used in the text of the present disclosure means the upper tissue layers, such as epidermis, dermis, adipose tissue, muscles, and deeper located fat tissue.

The term “adipose tissue” used herein may also encompass, fat, and other undesirable tissue elements. The term “adipose tissue” is an example of undesirable or excessive tissue, but it should also be understood that the processes and treatments disclosed are applicable to other classes of tissue.

The term “tissue treatment,” as used in the present disclosure means application of one or more types of energy to the tissue to alter the tissue, such as changing it to a different state, or obtain another desired treatment effect. The desired effect or state may include at least one of adipose tissue destruction, shrinking, breakdown, and skin tightening, haemostasis, inducing fat cells necrosis, inducing fat cells apoptosis, fat redistribution, adiposities (fat cell) size reduction, and cellulite treatment.

The terms “light,” “laser energy,” and “laser radiation” in the context of the present disclosure have the same meaning.

The term “tissue affecting energy” as used in the present disclosure means energy capable of causing a change in the tissue and/or skin or enabling such change. Such energy for example, may be RF energy from one or more areas in the electromagnetic spectrum, optical radiation in the visible or invisible part of electromagnetic spectrum, ultrasound waves energy, and kinetic energy provided by a massaging device.

The term “probe” as used in the present disclosure means any device operative to couple to the tissue or skin energy affecting the tissue/skin. Such device for example, may apply to the tissue RF energy, optical radiation existing in the visible or the invisible part of spectrum, energy from ultrasound waves, kinetic energy provided by a massaging device or some other source of energy.

As used herein, the term “subject” refers to any human or animal subject, as well as objects used to simulate the same for testing purposes.

As used herein, the term “treatment” means a process of coupling to the tissue or skin energy affecting the tissue/skin.

### BRIEF SUMMARY

A method and apparatus for adipose tissue treatment in which two types of electromagnetic radiation (or energy) are applied to a volume of tissue to be treated. One type of the electromagnetic energy is RF and the second type of electromagnetic energy is provided by visible or infrared radiation.

In some embodiments, both types of electromagnetic energy are delivered to the target volume subcutaneously by a light guide or needle that includes electrodes. In other embodiments, only one type of energy may be delivered to a target volume.

In some embodiments, the RF energy is delivered to a target volume of the tissue by an electrode applied to the skin. In other embodiments, the energy may be delivered to a target volume by two or more electrodes introduced subcutaneously into the tissue. The energy delivered by the visible or infrared radiation is delivered subcutaneously by a needle or probe, which is introduced into the same target volume of the tissue.

### BRIEF LIST OF DRAWINGS

The disclosure is provided by way of non-limiting examples only, with reference to the accompanying drawings, wherein:

FIG. 1 is a schematic illustration of the first exemplary embodiment of an electromagnetic energy-conveying needle.

FIGS. 2A-2C, collectively referred to as FIG. 2, are schematic illustrations of a number of cross sections of some of the exemplary embodiments of the needle of FIG. 1.

FIGS. 3A and 3B are schematic illustrations of a second exemplary embodiment of an electromagnetic energy-conveying needle.

FIGS. 4A-4C are schematic illustrations of a third exemplary embodiment of an electromagnetic laser energy-conveying needle.

FIGS. 5A-5C are schematic illustrations of a fourth exemplary embodiment of an electromagnetic energy-conveying needle.

FIGS. 6A-6C are schematic illustrations of a fifth exemplary embodiment of an electromagnetic energy-conveying needle.

FIGS. 7A-7C are schematic illustrations of a sixth exemplary embodiment of an electromagnetic energy-conveying needle.

FIGS. 8A through 8D are schematic and cross-section illustrations of a seventh exemplary embodiment of the electromagnetic energy-conveying needle and RF electrode configurations of the tip for a tissue suction probe cannula.

FIG. 9 is a schematic illustration of an eighth exemplary embodiment electromagnetic energy-conveying needle and RF electrode configurations the tip for a tissue suction probe cannula.

FIGS. 10A-10D are schematic illustrations of additional exemplary embodiments of an electromagnetic energy-conveying needle.

FIG. 11 is a schematic illustration of the ninth exemplary embodiment of an electromagnetic energy-conveying needle.

FIG. 12 is a schematic illustration of an exemplary embodiment of an apparatus for laser and RF assisted liposuction employing the present needle.

### DETAILED DESCRIPTION

The present disclosure presents features, aspects and elements that may be included in one or more embodiments of a needle or probe, apparatus and/or method. As a non-limiting example, one embodiment of the needle or probe includes a tip for a tissue suction probe. The exemplary tip may include a main lumen, a side lumen and an electrode. The main lumen may have an open end that can engage with a suction probe, and a closed end on the opposite side from the open end. In addition, the main lumen may one or more apertures adjacent or near to the closed end of the lumen. The apertures are defined by an edge or rim of the lumen. The first side-lumen extends at least partially along the side of the main lumen and traversing the closed end. The side-lumen may extend along the main lumen in a manner that relative to the main axis of the main lumen is a parallel path, a spiral path, an arbitrary path or some other fashion or combination thereof. The side-lumen extends through or communicates with an outlet located or defined in the closed end of the lumen. The side lumen may be fixedly or permanently attached to the main lumen or may be temporarily attached or removable. The electrodes are disposed along a portion of the outer surface of the tip and extend over a portion of rim. The electrodes also may extend to the inner surface of the lumen.

Additional embodiments may include more than one side-lumen with each side-lumen being configured or operative to carry and deliver a fluid, such as an irrigation fluid, and/or for extracting fluids from the application area. Some embodiments my include one, two, three or more RF electrodes. In the various embodiments, the RF electrodes are configured to induce an RF current between them when connected to a source of RF power, and to heat tissue traversing the aperture and entering into the main lumen of the tip. In other embodiments, multiple electrodes may be included on the tip and one or more of the electrodes can be selected or deselected, enabled or disabled, either manually or automatically. For instance, a switch may be used to enable/disable certain electrodes or groups of electrodes. Likewise, the tip may include sensors, such as capacitive switched to detect when a probe should be enabled or disabled.

The various embodiments may be used for applying electromagnetic radiation generated by one or more different electromagnetic radiation sources to a target volume of tissue. For example, in one application a source of electromagnetic radiation is applied externally so that the radiation penetrates the surface of the tissue and is concentrated in the target volume. A second source of electromagnetic radiation can then be applied to the same target volume by a second source located within the volume of tissue. In such an application, it is desirable to set the level of the first source such that it is insufficient to produce a desired treatment effect on its own. Then the energy level of the second source is set to a level that when combined with the first source, the combination is sufficient to produce a desired treatment effect. As a particular non-limiting example, in such an application the first source of energy may be RF radiation and the second source infrared radiation.

The principles and execution of the needle or probe, apparatus, and method described thereby may be best understood by reference to the drawings, wherein like reference numerals denote like elements through the several views and the accompanying description of non-limiting, exemplary embodiments.

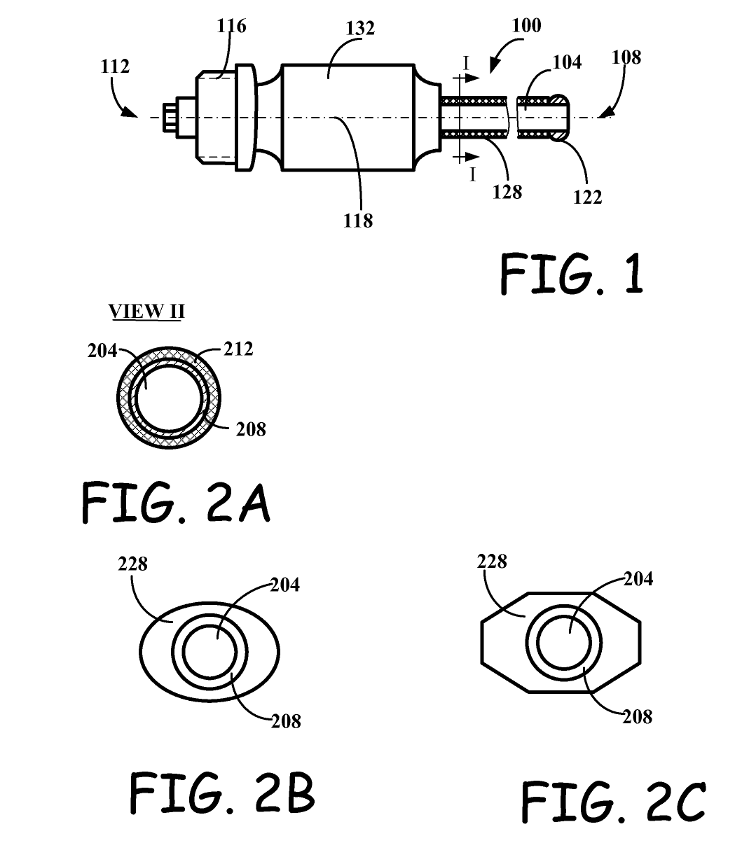

Reference is made to FIG. 1, which is a schematic illustration of a first exemplary embodiment of an electromagnetic radiation-conveying needle. Needle or probe 100 is a needle shaped solid or hollow light conducting guide 104 having a first 108 end and a second end 112. First end 108 can be shaped for piercing the skin of a subject (not shown). The second end 112 is adapted to connect directly to a source of laser radiation by means of a connector (not shown) similar to a fiber optics type connector, for example SMA type connector and additional cable. Adjacent to first end 108 of needle 100 a mono-polar RF (Radio Frequency) electrode 122 is located and connected through the same connector 116 to a source of RF energy (not shown), which is a type of electromagnetic energy. Electrode 122 may connect to the source of RF energy, operating in frequency range of 100 KHz to 100 MHz, by a conventional conductive wire or specially deposited leads terminating at connector 116 over which for isolation purposes a protective coating or jacket 128 may be placed. Electrode 122 may be a thin metal sleeve or a ring having rounded angles stretched over first end 108 of needle 100 and fixed by any known means. The length of electrode 122 may be 1 to 50 millimeter depending on the type of treatment applied. Alternatively, electrode 122 may be electrochemically deposited on first end 108 of needle 100. Electrode 122 may be located adjacent to the first end of needle 100 such that first end 108 of needle 100 would protrude from electrode 122 or reside inside electrode 122.

First end 108 of needle 100 may be shaped for piercing the skin of a subject and may be terminated by a plane perpendicular to the optical axis 118 or at an angle to the optical axis 118 of needle 100. Alternatively, end 108 may have a radius or an obtuse angle. Other shapes of needle end 108 that improve either subject skin penetration properties, facilitate needle or probe movement inside fibrotic fatty tissue, or laser power delivery quality are possible. In some cases, the skin incision is made by any well-known surgical means and the needle is introduced into the tissue. In an alternative embodiment laser radiation emitted through the first end 108 of needle 100, assists needle 100 into skin penetration process by providing continuous or pulsed laser power suitable for skin incision. Numeral 132 designates a handle by which the caregiver or person providing treatment holds and operates the needle. Handle 132 may include certain knobs for initiating or terminating treatment related processes. The length of needle 100 may vary from a few millimeters to a few hundred millimeters.

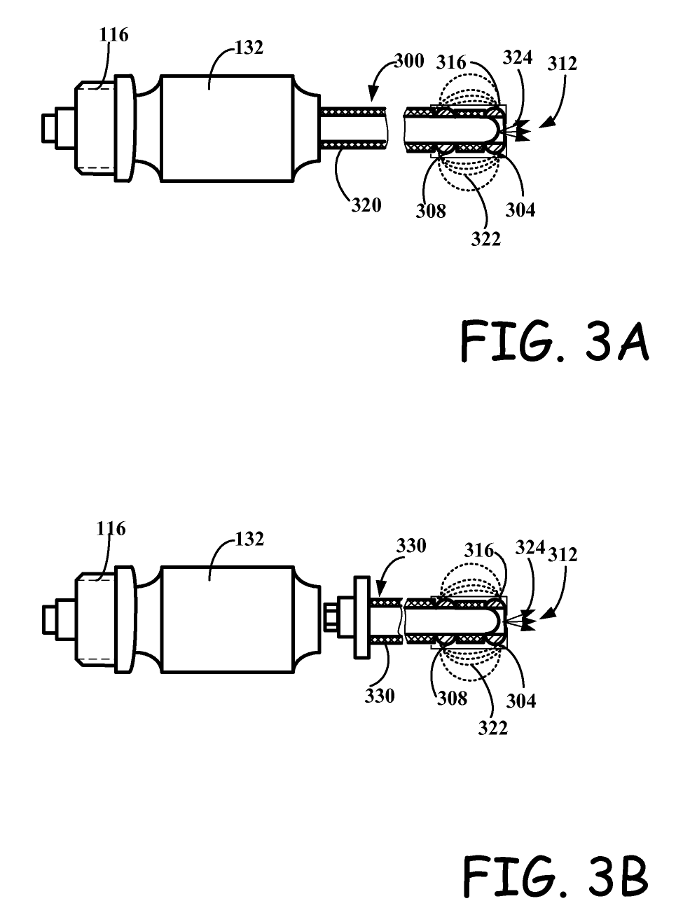

FIG. 2A is an exemplary cross section of needle 100 that has a round cross section. Needle 100 includes a solid light conducting core 204, a cladding 208 having a refractive index lower than core 204, and a protective jacket 212 that mechanically protects the sensitive surface of the needle. The diameter of core 204 may be 100 micron to 1500 micron, the diameter of cladding 208 may be 110 micron to 2000 micron, and the size of jacket 212 may be 200 micron to 2500 micron. Connection of needle body 104 to connector 116 may be performed by crimping or any other means known and established in the fiber optics industry.

In some embodiments, shown in FIGS. 2B and 2C, jacket 228 may have an elliptical or polygonal shape. These shapes provide different stiffness along the short and long symmetry axes of the needle cross section, and facilitate introduction and movement of the needle into the subject body.

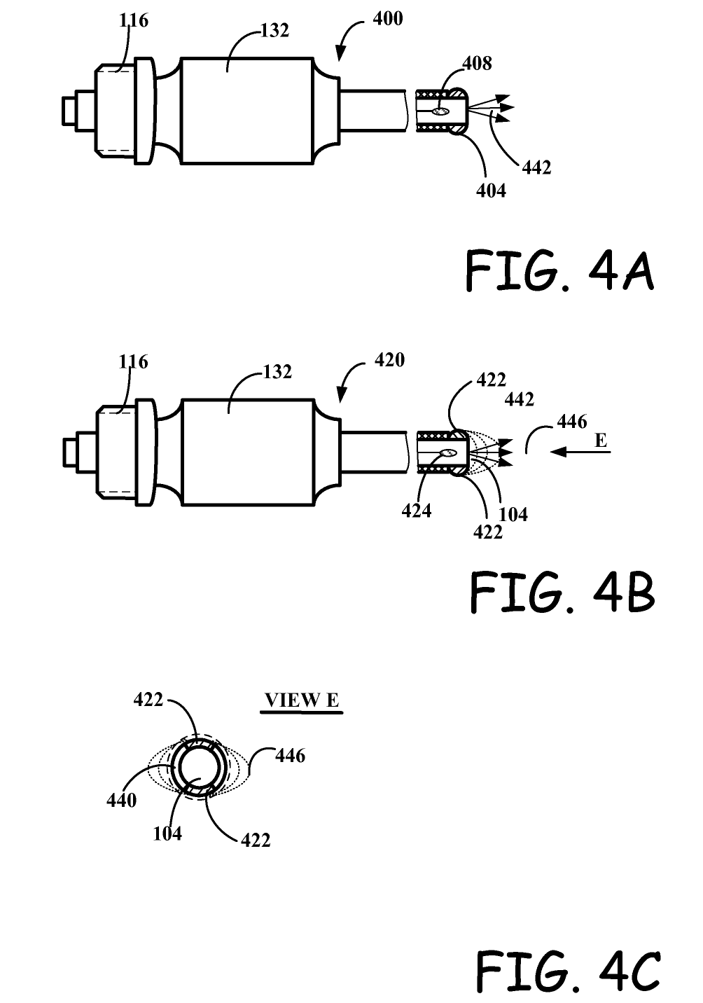

FIG. 3A and 3B collectively termed FIG. 3 are a schematic illustration of a second exemplary embodiment of an electromagnetic energy-conveying needle. It illustrates a needle or probe 300 with bipolar electrodes 304 and 308 located adjacent radiation or energy emitting end 312 of needle 300. Electrodes 304 and 308 may be in a conductive coupling with the tissue of the treated subject or may be coated by a dielectric layer 316 and be in a capacitive coupling with the treated subject tissue. Electrodes 304 and 308 may be produced in a way similar to the one described above. FIG. 3 shows an exemplary embodiment of needle 300 with laser radiation emitting end 312 implemented as a spherical end. Other laser radiation emitting end 312 terminations are possible. Numeral 320 marks the fiber optics guide jacket. FIG. 3A illustrates a disposable or reusable needle 300 that includes handle 132. FIG. 3B illustrates a disposable or reusable needle 330 that in use is attached to handle 132. Numeral 322 marks RF current and numeral 324 marks the emitted laser radiation.

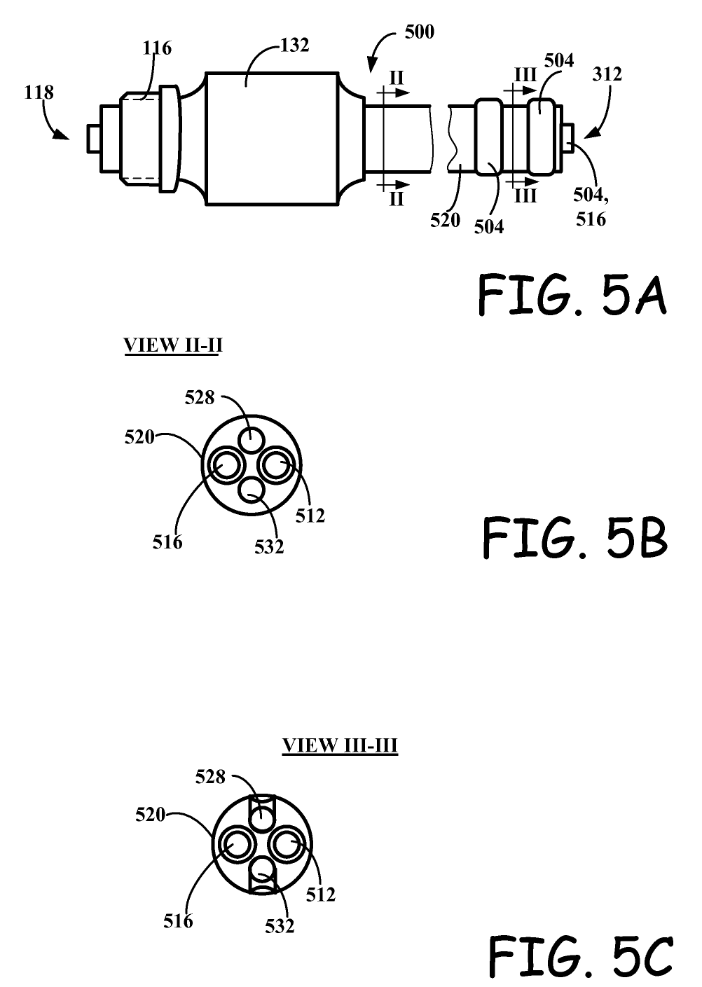

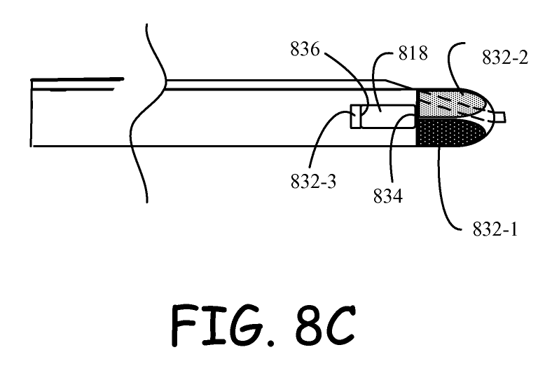

FIG. 4 is a schematic illustration of a third exemplary embodiment of an electromagnetic radiation-conveying needle. Needle or probe 400 (FIG. 4A) includes a mono-polar electrode 404 and a temperature sensor 408 that measures temperature in the target tissue volume. Knowledge of the temperature in the target tissue volume helps in informing a caregiver on the treatment status and in establishing proper feedback to controller 818 (FIG. 8) and setting appropriate treatment parameters.

FIG. 4B is an illustration of a needle 420 with two electrodes 422 and temperature sensor 424. Electrode 404 (mono-polar) or electrodes 422 (bi-polar) may be implemented as one or more conductive rings or as a film deposited on one or both (opposite) sides of needle 420 circumference. Lines 446 indicate the current induced by bi-polar electrodes in the tissue and numeral 442 marks emitted by the needle laser radiation.

FIG. 4C is a view illustrating the radiation-emitting end of needle 420 with bi-polar electrodes 422 at least partially conforming to the needle shape. The electrodes may be made of foil, wire, thin metal plates, or electrochemically deposited. A temperature sensor 424 may also be placed on guide 104. An optional layer of a dielectric or isolator to avoid crosstalk or potential short circuit between the electrodes may coat the electrodes. Numeral 440 marks isolation between electrodes 422, which may be part of the dielectric coating or similar material. Changing the size of electrodes, (the size of the segment conforming to the needle shape) allows the volume of affected RF tissue to be changed.

In a bi-polar RF electrode configuration, an additional treatment progress status feedback method may be implemented. When RF energy is supplied to electrodes 422 it induces a current flow shown schematically by phantom lines 446 in the tissue between electrodes. It is known that tissue conductivity is temperature dependent. Accordingly, measuring the RF induced current value provides information on treated tissue status and allows the power and time of each of the laser radiation 442 or RF energy supplied to the target skin/tissue volume to be regulated.

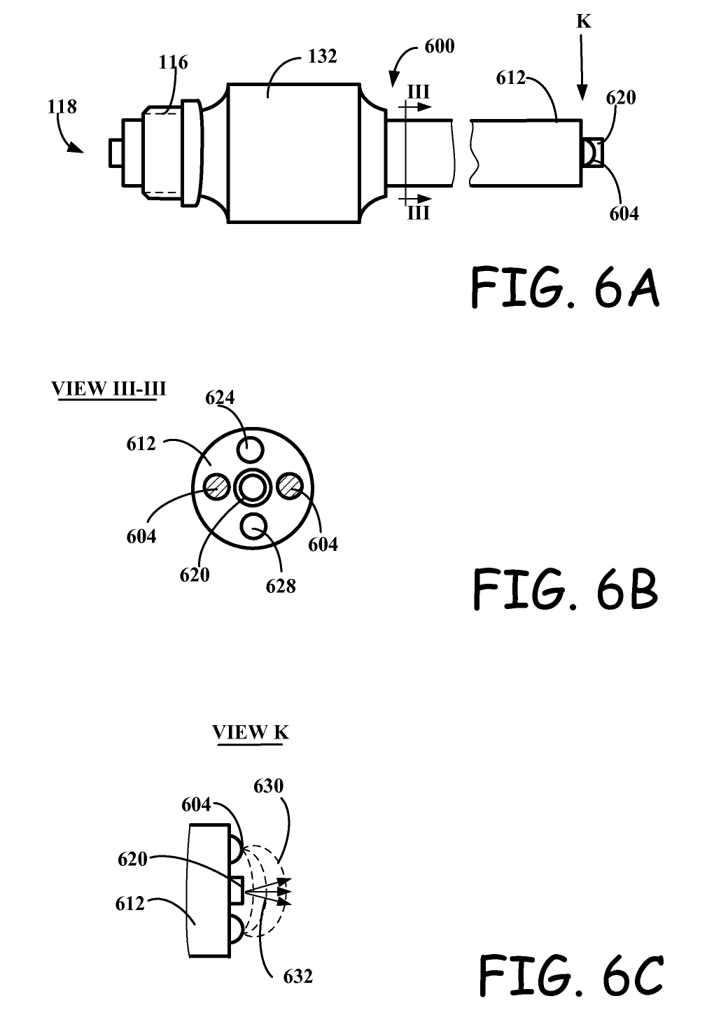

FIG. 5A is a schematic illustration of a fourth exemplary embodiment of an energy-conveying needle or probe 500 with RF energy supplying electrodes 504 and two light conducting guides 512 and 516. Both the RF energy-supplying electrodes 504 and light conducting guides 512 and 516 are incorporated into a connecting member 520 forming a single catheter like structure. RF electrodes 504, which may be rings of biocompatible conductive material, are tightened or deposited over the connecting member 520, which may be made from isolating material. One or more fluid conducting channels 528 and 532 may be made in connecting member 520. For example, fluids delivered through fluid delivery channel 528 may be used for cooling or heating the electrodes, or any other desired part of the needle or tissue, conductive fluids may be introduced into the treated tissue volume through channel 528. Other fluids may also be delivered through channel 528. Fluids may also be delivered for irrigation purposes. In such embodiments, the irrigation fluids may be delivered in such a manner so as to displace or distant tissue from the tip of said light guide fiber. For instance, as non-limiting examples, the fluid may be delivered in a volume and/or at a pressure sufficient to displace such tissue. Further, the fluid may include antiseptics, Novocain, hydrogen peroxide, or other chemicals or medications to assist in the treatment. Further, the fluid may be delivered in such a manner to prevent charring of the tip of said light guide fiber. Again, this may include, as non-limiting examples, providing the fluid with sufficient volume and/or pressure and/or of such composition to prevent or limit the charring. As such, in such embodiments the fluid delivery channels may be connected to a source of irrigation fluid. Adipose tissue treatment products and the fluid supplied to the tissue may be removed through fluid removal channel 532. In some embodiments, their may be one fluid conducting channel only and it may be used either for different fluids delivery to the treated volume or adipose tissue treatment products removal. There may be a switching arrangement switching as required the same channel between the two processes.

Channel 532 connects to a facility for adipose tissue laser treatment products removal 824 (FIG. 8A) and the fluid delivery channel 528 is connected to a source of fluid delivered through the lumen of probe 820 (see FIG. 8) with the help of the same connector 116 or by a separate connector. Operation of the facility for adipose tissue laser treatment products removal and the source of fluid synchronize with the operation of laser source and RF energy delivery.

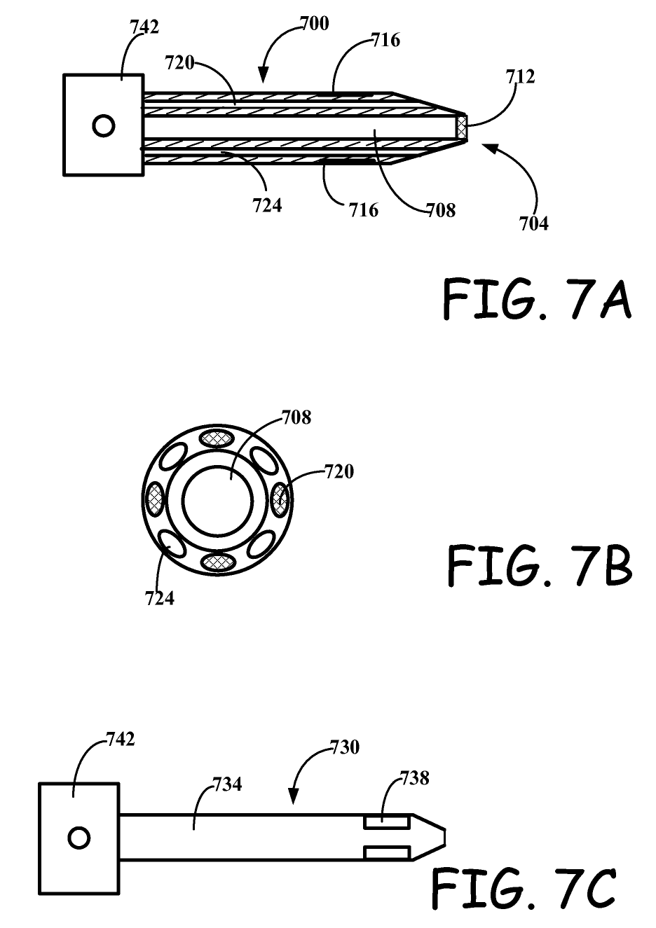

FIGS. 6A and 6B are schematic illustrations of a fifth exemplary embodiment of an energy-conveying needle with RF energy supplying electrodes. Needle or probe 600 contains two, rod type electrodes 604, a light conducting guide 620, a fluid delivery channel 624 and adipose tissue treatment products removal channel 628, all incorporated into a common catheter-like structure 612. Light conducting guide 620 is connected to a source of laser radiation of suitable wavelength and power. If necessary, fluid may be supplied to the target volume (not shown) through delivery channel 624. Adipose tissue treatment products such as liquefied fat, if necessary, may be removed through removal channel 628. FIG. 6C illustrates operation of probe 600. Numeral 630 illustrates RF current lines and numeral 632, laser radiation irradiating the target tissue volume.

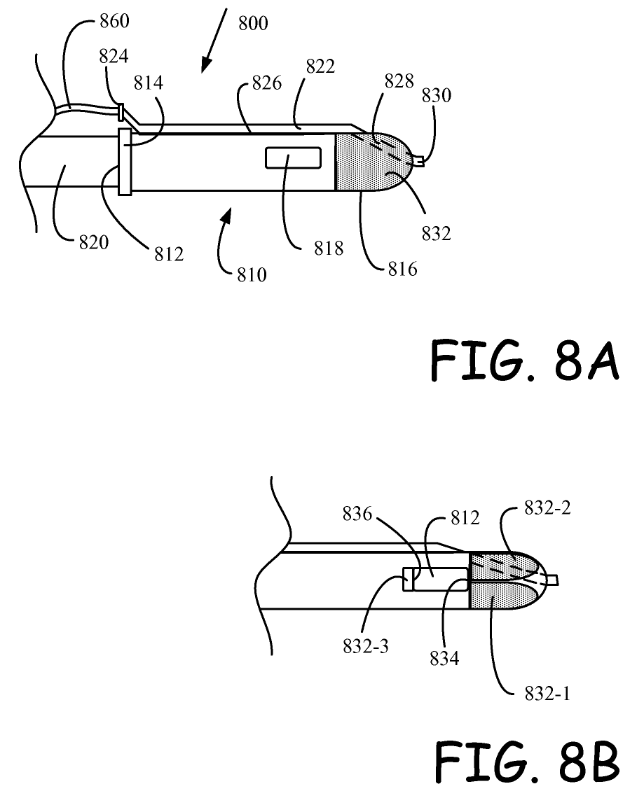

FIG. 7 is a schematic illustration of a sixth exemplary embodiment of a flexible or rigid, hollow or solid energy-conveying needle or probe 700. The emitting end 704 of light guide 708, which is introduced into the adipose tissue for treatment, is covered by a sapphire, diamond, or YAG window 712. During the course of liquefying adipose tissue, certain materials (termed carbonized materials) resulting from tissue with RF energy and high laser power interaction, deposit on end 708 of needle 700. These carbonized deposits increase laser light absorption by end 708 of needle 700 reducing the amount of laser radiation delivered to the target tissue volume. This deposit should be removed periodically. Increased laser power absorption in the carbonized deposit can increase local temperature at the first end 712 of needle 700 resulting in the needle damage. Sapphire, YAG, and diamond or similar materials are generally resistant to high temperature. Their use as a termination of the first end of the needle significantly improves the carbonization resistance and useful life of the needle.

Similar to the earlier disclosed exemplary embodiments, needle 700 includes one or more electrodes 716 deposited or built-in into the external surface of the needle. As shown in FIG. 7B, needle or probe 700 may have channels 720 for fluid supply and channels 724 for liquefied fat and other adipose tissue laser treatment products removal and aspiration. In some embodiments, their may be one fluid conducting channel only and it may be used either for fluid delivery or adipose tissue treatment products removal.

FIG. 7C is an illustration of a needle 730, the body 734 of which is made completely of sapphire. Such a needle is more resistant than glass needles to deposition of carbonized laser treatment products. Electrodes 738 conforming to the shape of needle 730 may be incorporated in needle 730. A protective and insulating layer may cover the electrodes if necessary. Needles 700 and 730 may connect by their second end 742 with the help of an additional cable to a controller 818 (FIG. 8) or similar

Referring now to FIGS. 8A through 8D, which are schematic and cross-section illustrations of a seventh exemplary embodiment of an electromagnetic energy-conveying needle and RF electrode configurations of the tip for a tissue suction probe cannula.

As shown in FIG. 8A, a probe for liposuction 800 may include a probe 820, similar to probe 100 of FIG. 1 or 300 of FIG. 3A or any other described above probe, removably or integrally attached to a tip 810. Tip 810, may be multi-use or disposable, tubular in shape, have a fluid and tissue removal lumen 850 (FIG. 8D) and an open end 812 engageable with probe 820 or another type of suction probe via a connector 814. When engaged, lumen 850 communicates with the lumen of probe 820 or that of another type of suction probe. The end of tip 810 opposite to open end 812 is closed, commonly by a dome-shaped closure 816. Tip 810 also includes one or more apertures 818 adjacent to closed end 816 and communicating with lumen 850.

A side channel 822 extends from an inlet port 824 outside of tip 810 along the entire length of tip 810, along outer surface 826 and is fixedly attached thereto as by a suitable adhesive, through wall 828 to an outlet 830. Channel 822 may be operative to slidingly accommodate one or more light guide fibers 860 threaded through inlet port 824 and exiting and protruding from outlet 830.

In the embodiment shown in FIG. 8A, an RF electrode 832 is disposed along the outer surface of dome-shaped closure 816 in a monopolar configuration. In this configuration, RF induced current affects the tissue area/volume that is proximate to the active electrode.

FIGS. 8B, 8C and 8D illustrate three RF electrode configurations of another exemplary embodiment of the tip for a tissue suction probe. In this embodiment, a rim portion 834 of aperture 818 abuts dome-shaped closure 816.

As shown in FIGS. 8B, 8C and 8D, tip 810 includes a tri-RF electrode configuration in which electrodes 832-1 and 832-2 are disposed along the outer surface of dome-shaped closure 816 and electrode 832-3 is disposed along a rim portion 836 of aperture 818, opposite rim portion 834.

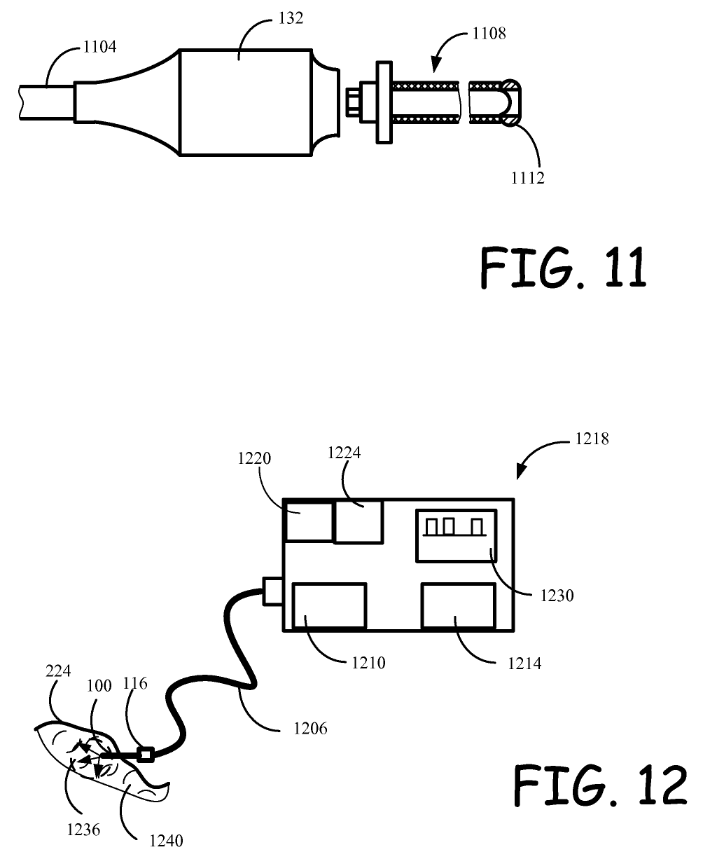

FIG. 8B depicts another monopolar electrode configuration, similar to that in FIG. 8A in which RF electrodes 832-1 and 832-2 when in use may be short circuited by controller 1218 (FIG. 12) or supplied by the same RF voltage and act as an active electrode, whereas RF electrode 832-3 operates as an inactive or passive electrode. In this configuration, a passive electrode (not shown) is coupled to the subject and acts as a grounding electrode. Typically, the electrode is different in size from electrodes 832-1 and 832-2 and is located at a distance from the active electrodes. In this configuration RF induced current affects the tissue area/volume that is proximate to the active electrodes.

FIG. 8C illustrates a bipolar RF electrode configuration of tip 810. The electrode placement configuration shown in FIG. 8C is similar to that of FIG. 8B but in this configuration electrodes 832-1 and 832-2 are operative electrodes. As in FIG. 8B, here too electrode 832-3 is inactive.

In this configuration, the area/volume of tissue to be treated and the propagation of the current is limited to the area between electrodes 832-1 and 832-2.

In the configuration illustrated in FIG. 8D, which is a schematic and cross-section illustration of another electrode configuration of the tip for a tissue suction probe, electrodes 832-1 and 832-2 are short-circuited whereas electrode 832-3 becomes an active electrode operating in bipolar configuration. Electrodes 832-1 and 832-2 may, in this configuration, be disposed along a portion of the outer surface of said dome-shaped closure 816, extend over aperture 818 rim portion 834 through aperture 818 opening and along a portion of an inner surface 838 of closure 816.

This results in a flow of current, as depicted by broken-line arrows 870, across aperture 818 heating and liquefying any adipose tissue entering lumen 850 through aperture 818 as depicted by the arrow designated reference numeral 872.

During the procedure, the operator may manually select any one of the aforementioned electrode charge configurations, as necessary. Alternatively, the selection of the electrode charge configuration may be controlled by a controller, such as controller 1218 (see FIG. 12) in accordance with the operator's input or a predetermined treatment protocol.

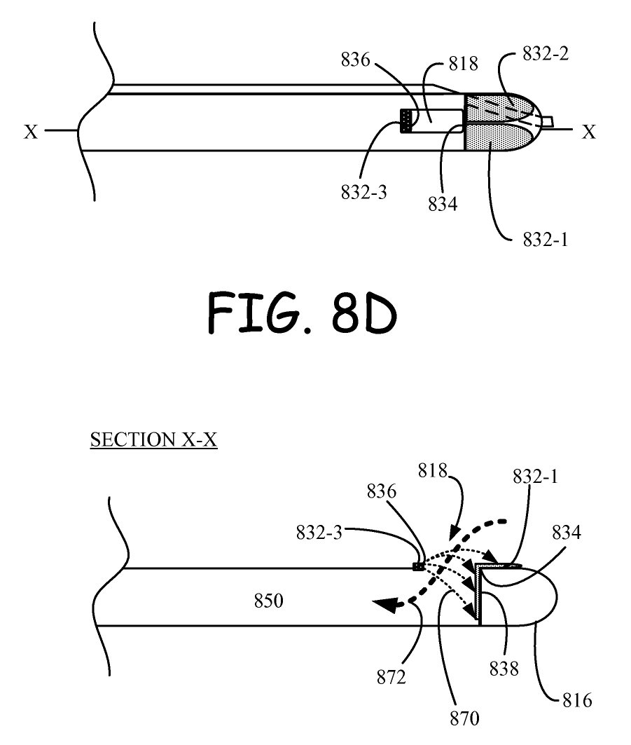

Referring now to FIG. 9, which is another exemplary embodiment of electromagnetic energy-conveying needle and RF electrode configurations the tip for a tissue suction probe. Tip 910 includes a side channel 922, which extends from an inlet port 924 outside of tip 910 along the entire length of tip 910, along outer surface 926 and is fixedly attached thereto as by a suitable adhesive, through wall 928 of closure 916 to an outlet 930. Channel 922 may be operative to slidingly accommodate a light guide fiber 960 threaded through inlet port 924 and exiting and protruding from outlet 930.

A fluid delivery channel 932 extends from an inlet port 934 outside of tip 910 along the entire length of tip 910, along outer surface 926 and is fixedly attached thereto by a suitable adhesive, through wall 928 of closure 916 to an outlet 940. Channel 932 may be operative to connect via port 934 to a fluid supply line 962 supplying fluid from a fluid source (not shown). The fluid supplied through port 934, delivered via channel 932 and ejected through outlet 940 may be employed for cooling the electrodes, or any other desired part of the tip or tissue. Tumescent fluids may also be introduced into the treated tissue volume through lumen or inlet port 934 as well as other fluids. Adipose tissue treatment products and the fluid supplied to the tissue may be removed through aperture 918 and fluid and tissue removal lumen 950. In some embodiments, there may be one fluid conducting channel only and it may be used either for delivery of various fluids to the treated volume or for adipose tissue treatment products removal. There may be a switching arrangement switching as required the same channel between the two processes including valve switching or other similar technique

Lumen 850 (FIG. 8D) connects to a facility for adipose tissue laser treatment products removal (not shown) and fluid delivery channel 932 may be connected to a source of fluid (not shown) via port 934. Operation of the facility for adipose tissue laser treatment products removal and the source of fluid may be synchronized with the operation of laser source and RF energy delivery.

The fluid delivered by fluid delivery channel 932 may also be employed to distant tissue from the tip of light guide fiber 960 to prevent carbonization or charring thereof. Alternatively or additionally, the fluid may be employed to lavage/irrigate the tissue being treated.

In accordance with another embodiment of the current tip for a tissue suction probe, tip 910 may include a dome-shaped shield 950 operative to protect the tip of light guide fiber 960 from carbonization or charring. Shield 950 may be integrally or removably attached by a screw-on, snap-on or similar type system to closure 916 thereby covering outlet 930. Alternatively, shield 950 may be integrally or removably attached to outlet 930. Shield 950 may be made of one or more materials selected from a group of glass, sapphire, quartz and other transparent heat resistant materials.

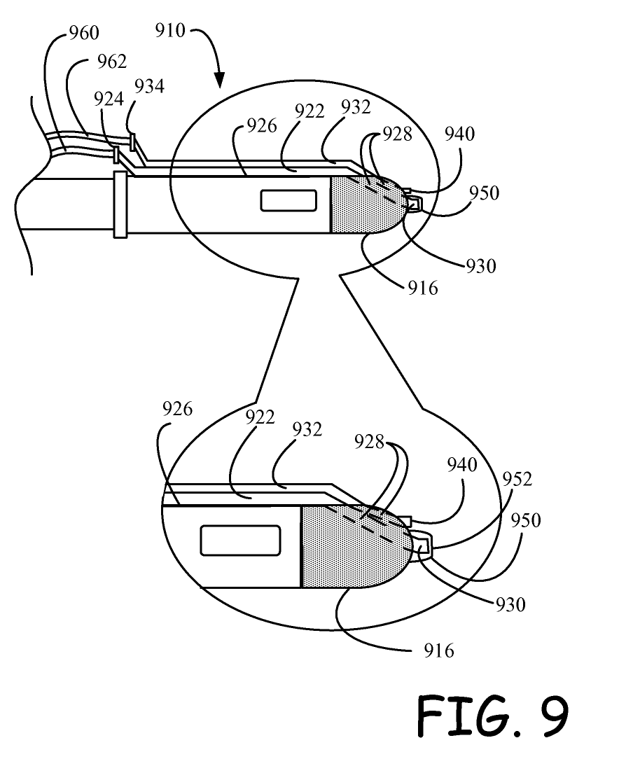

FIGS. 10A-10D are schematic illustrations of additional exemplary embodiments of the needle for laser and RF assisted liposuction. FIG. 10A illustrates a needle or probe 1000 having a jacket 1002 and a light conducting body 1004 made from electrically non-conductive material. A cylindrical electrode 1006 is drawn over the radiation or energy-emitting end 1008, of light conducting body 1004. A cylindrical bushing 1010 having a proximal end 1012 and a distal end 1014 is tightly fit over the light conducting body 1004 or over jacket 1002. Distal end 1014 of bushing 1010 is formed to receive a second electrode 1016. Both electrodes, which may be concentric and coaxial electrodes, are connected to the source of RF energy 1214 (FIG. 12). Bushing 1010 features one or more openings 1018 arranged on opposite sides of bushing 1010. As needle 1000 moves back and forth, it picks-up new portions of RF heated fat tissue, the flow of which is shown by lines 1022. Lines 1026 illustrate RF induced current and lines 1028 illustrate schematically the laser radiation melting the fat. Laser radiation 1028 is emitted into the fat volume located between electrodes 1006 and 1016 in a pulse and/or continuous radiation mode. In some embodiments, either a pulse mode or continuous mode is utilized but in some embodiments, both modes can be utilized and selected under user control or based on algorithmic or programmed decisions or heuristics. The laser radition provides additional energy for faster fat liquefaction. Needle 1000 may include fluid conducting channels (not shown) for delivery or removal of fluids such as a cooling fluid, heating fluid, conductivity changing fluid, or products of adipose tissue treatment.

FIG. 10B illustrates a needle or probe 1030 including a protruding light guide 1032 and electrode 1034 having a shape that is easier to advance in a path formed in the adipose tissue by laser energy emitted through the end of light guide 1032. Needle 1030 may include fluid conducting channels (not shown) for delivery or removal of fluids such as a cooling fluid, heating fluid, conductivity changing fluid, or products of adipose tissue treatment.

FIG. 10C illustrates a needle 1040 comprising a light guide 1042 made from electrically non-conductive material or a layer of isolation placed over light guide 1042. The first end 1044 of needle 1040 is formed to enable laser radiation 1046 emissions in the direction of target volume 1050. The first end 1044 may include multiple holes or a single hole for allowing the laser radiation to exit towards the target volume 1050. Lines 1048 indicate RF induced current heating a target volume 1050 of the tissue. Laser radiation 1046 is emitted into the same target volume 1050 that is heated by the RF induced current in a pulse or continuous radiation mode and provides additional energy for faster fat liquefaction. Electrodes 1052 and 1054 may be coated by a dielectric or be in direct contact with the tissue. An extender 1026 may be attached to needle 1040 for mounting electrode 1054 on it. Alternatively, electrode 1054 may be attached directly to needle 1040.

FIG. 10D illustrates a needle 1060 including a light conducting body 1064, the first end 1068 which is shaped to generate a certain radiation distribution pattern illustrated by arrows 1070 or diffuse laser power uniformly at the target treatment volume. The radiation-diffusing end would typically be 3 mm to 30 mm and such needle may be used, for example, at high laser power to avoid local overheating and needle tip carbonization. Needle 1060 may be used for haemostasis.

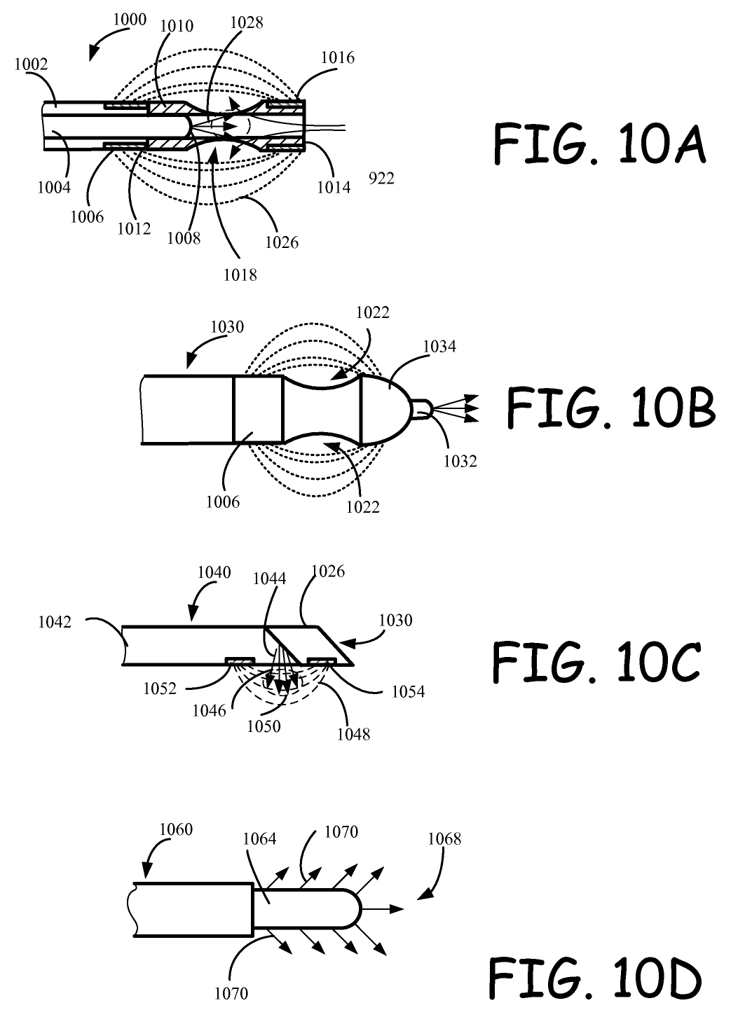

FIG. 11 is a schematic illustration of a ninth exemplary embodiment of a laser radiation-conveying needle, which may be a disposable or reusable needle. Handle 132 (FIG. 1) is integral with an interim light guide, which is incorporated into cable 1104, and needle 1108 is implemented as a reusable/exchangeable or disposable part. Cable 1104 may include one or more fluid supply channels and/or one or more treated tissue debris removal channels. Relevant conductors supplying RF energy to electrodes 1112 could be incorporated in cable 1104. The disposable part 1108 may be connected to handle 132 by any known and suitable quick connection/removal connectors. Any one of the similar needle structures described herein could be used instead of disposable needle 1108.

FIG. 12 is a schematic illustration of an apparatus for laser and RF assisted liposuction suitable for using one or more of the described needle embodiments. Connector 116 connects needle 100 or 300 or any other needle described above via a cable 1206 to a source of laser radiation 1210 and a source of RF energy 1214, which may be incorporated into a controller 1218, or possibly stand-alone units. In addition, cable 1206 may include at least one fluid conducting channel connecting the needle to a source of fluid 1220 and/or adipose tissue treatment products removal facility 1224.

In some embodiments, the needle is long enough to connect directly to a source of laser radiation 1210 and a source of RF energy 1214. In such case, a separate cable (not illustrated) may include the RF conducting leads, which connect electrodes directly to the controller. Cooling fluid conducting and removal channels may be included in either of the cables. Controller 1218 may operate the source of laser radiation 810 and the source of RF energy in a pulse or continuous radiation mode.

Controller 1218 may further include a display 1230 with a touch screen, or a set of buttons or actuators providing a user interface and synchronizing operation of the source of laser radiation 1210 and the RF generator 1214 with the operation of facility for adipose tissue treatment products removal facility 1224 and a source of fluid 1220.

When RF energy of proper value is applied to the adipose tissue, it heats the tissue and may liquefy it. Laser radiation of proper power and wavelength when applied to the adipose tissue may destroy fibrotic pockets releasing liquefied fat. The liquefied adipose tissue may be removed or may be left in the body, where it gradually dissipates. Application of each of the energies alone requires a significant amount of energy, which is associated with high cost. Generally, the energy provided by laser radiation is more costly than that of RF energy.

The present apparatus enables a method for adipose tissue laser treatment combining the RF energy and laser radiation. For treatment, needle 100 or any other needle described above is introduced into a target tissue volume 1236 of adipose tissue 1240. RF generator becomes operative to supply lower cost RF energy to the target volume and heat it to a desired temperature. A relatively small addition of laser energy or radiation is required to liquefy target volume of adipose tissue 1236, destroy fibrotic pockets and release the liquefied fat. Both the RF energy and laser radiation may be delivered into the target tissue volume in a pulse or continuous mode and either simultaneously or subsequently in at least partially overlapping periods of time. RF energy delivered to the target tissue volume 1236 heats the volume and laser radiation source 1210 delivers additional tissue-destroying energy to target volume 1236. Both laser and RF energies may cause controllable dermal collagen heating and stimulation.

Concurrently with the operation of the source of RF energy 1214 and laser radiation source 1210, the facility for adipose tissue treatment products removal 1224 and, if necessary, fluid supply facility 1220 become operative. The caregiver or apparatus operator moves the needle inserted in the tissue back and forth and periodically changes its angle of movement.

It is known that a number of wavelengths may be conducted through the same light guide. In order to facilitate the process of treatment location observation of tissue, an additional second laser, visible through skin/tissue laser, such as a HeNe laser may be coupled to needle 100 or cable 1206. The HeNe laser, which is visible through skin, may assist the caregiver/operator in repositioning first end 108 of needle 100 (FIG. 1). Upon completion of treatment, needle 100 may be discarded. In an alternative embodiment, a temperature sensitive cream or temperature sensitive liquid crystal paste or film may be applied to the skin 224 over the treated adipose tissue section. The paste/spread may be such as Chromazone ink commercially available from Liquid Crystal Resources/Hallcrest, Inc. Glenview Ill. 60026 U.S.A.

In yet another embodiment, laser beams from two laser sources with different wavelength could be used to optimize simultaneous fat destruction and blood haemostatis. The laser wavelengths may, for example, be 1,06 micrometer wavelength provided by NdYAG laser and a 0.9 micrometer wavelength provided by a laser diode. Another suitable set of wavelength is 1.064 micron and 0.532 micron. Such combination of laser wavelength reduces the bleeding, makes the fat removal procedure safer, and shortens the patient recovery time.

In still a further embodiment, following tissue heating or almost simultaneously with tissue heating by RF energy, a pulsed IR laser, for example a Ho—Tm (Holmium-Thulium) or Er:Yag laser generating pulses in sub-millisecond or millisecond range, may be applied to the same target tissue volume 1236. During the laser pulse, the target tissue (cells and intercellular fluid) near the end 108 (FIG. 1) of needle 100 (or any other needle end) changes to overheated (high-pressure) gas forming expanding micro bubbles collapsing at the end of the pulse. Mechanical stress developed by that action may increase the rate of membrane of adipose cell disruption and release of liquefied fat from the cell. This opto-mechanical action of laser radiation combined with volumetric RF heating efficiently liquefies fat and makes fat removal/suction more efficient. The laser radiation pulse induces mechanical stress on cells in the target volume and delivers additional energy to the target volume that is sufficient for adipose tissue destruction.

The apparatus disclosed above may also be used for skin tightening. The needle is inserted subcutaneously into a patient so that the first end of the fiber is introduced within the tissue underlying the dermis. RF energy and laser source emit radiation of suitable power that are conveyed by the needle and the electrodes to the dermis, where the radiation causes collagen destruction and shrinkage within the treatment area.

The disposable needle described enables continuous adipose tissue treatment process, significantly reduces the treatment time, makes the subject treatment more comfortable and simplifies the treatment process.

While the exemplary embodiment of the needle, apparatus and the method of treatment has been illustrated and described, it will be appreciated that various changes can be made therein without affecting the spirit and scope of the needle, apparatus or method of treatment. The scope of the needle, apparatus and the method of treatment therefore, are defined by reference to the following claims:

What is claimed is:

1. A tip for a tissue suction probe, said tip comprising:
a main lumen having an open end engageable with a suction probe, a closed end opposite to said open end and a rim defining at least one aperture adjacent to said closed end communicating with a lumen of said suction probe ;
a first side-lumen operative to slidingly accommodate a light guide fiber, said first side-lumen extending partially along said main lumen, traversing said closed end and communicating with an outlet located in said closed end; and
a first electrode and a second electrode disposed along a portion of the outer surface of said tip, extending over a first portion of said rim and abutting said closed end, through said aperture and along a portion of an inner surface of said closed end.

2. The tip for a tissue suction probe according to claim 1, wherein also comprising a second side-lumen configured to carry and deliver an irrigation fluid, said second side-lumen extending partially along said main lumen and said first side-lumen, traversing said closed end and communicating with a second outlet located in said closed end.

3. The tip for a tissue suction probe according to claim 2, wherein said first and second electrodes are short-circuited.

4. The tip for a tissue suction probe according to claim 2, further comprising a third RF electrode and wherein said first and second electrodes and said third electrode are configured to induce an RF current between them when connected to a source of RF power, and to heat tissue traversing said aperture and entering into the main lumen of said tip.

5. The tip for a tissue suction probe according to claim 2, further comprising a third RF electrode, wherein said electrodes may be may be selected manually or automatically.

6. The tip for a tissue suction probe according to claim 1, wherein also comprising a second side-lumen extending partially along said main lumen, extending beyond said closed end and said first side-lumen engageable with a source of irrigation fluid and configured to carry and deliver an irrigation fluid.

7. The tip for a tissue suction probe according to claim 6, wherein said irrigation fluid is delivered in a manner to distant tissue from the tip of said light guide fiber.

8. The tip for a tissue suction probe according to claim 6, wherein said fluid is delivered in a manner to prevent charring of the tip of said light guide fiber.

9. The tip for a tissue suction probe according to claim 6, wherein said first side-lumen and second side-lumen are fixedly attached to said main lumen.

10. The tip for a tissue suction probe according to claim 6, wherein said first side-lumen and second side-lumen are removably attached to said main lumen.

11. The tip for a tissue suction probe according to claim 1, wherein said first side-lumen and second side-lumen extend partially along said main lumen following a path in a manner such that at least a portion of the path consists of at least one of a parallel path, a spiral path and an arbitrary path relative to the main axis of said main lumen.

12. The tip for a tissue suction probe according to claim 1, wherein said first and second electrodes are RF electrodes and configured to form an RF field and induce a current between the electrodes and heat tissue outside and surrounding said tip when said first and second electrodes are connected to a source of RF energy.

13. The tip for a tissue suction probe according to claim 1, wherein said open end of said tip is integrally attached to said suction probe.

14. The tip for a tissue suction probe according to claim 1, further comprising a shield, removably attached to said outlet, and operative to accommodate the end of a light guide fiber.

15. The tip for a tissue suction probe according to claim 14, wherein said shield is made of at least one material selected from a group consisting of glass, sapphire, quartz, and other transparent heat resistant materials.

16. The tip for a tissue suction probe according to claim 1, wherein said tip is disposable.

17. An apparatus for tissue treatment, said apparatus comprising:
a tip for a tissue suction probe comprising:
a main lumen having an open end engageable with a suction probe, a closed end opposite to said open end and a rim defining at least one aperture adjacent to said closed end communicating with a lumen of said suction probe ;
a first side-lumen configured to slidingly accommodate a light guide fiber, said first side-lumen extending partially along said main lumen, traversing said closed end and communicating with an outlet located in said closed end; and
at least one electrode disposed along a portion of the outer surface of said tip, extending over a first portion of said rim and abutting said closed end, through said aperture and along a portion of an inner surface of said closed end;
one or more sources of laser energy that can be coupled to said tip; and
a source of RF energy operatively configured to provide RF energy to the at least one electrode.

18. The apparatus according to claim 17, wherein said one or more sources of laser energy is configured to provide laser energy in at least one mode including a pulse mode and a continuous energy-emitting mode.

19. The apparatus according to claim 17, wherein said source of RF energy provides the RF energy to said at least one electrode in at least one mode including a pulse mode and a continuous energy delivering mode.

20. The apparatus according to claim 17, wherein the tip further comprises at least one fluid-conducting lumen.

21. The apparatus according to claim 17, wherein the tip further comprises at least one fluid-conducting lumen and the apparatus further comprises a controller , said controller being configured to synchronize the operation of said one or more sources of laser radiation, the source of RF energy, and a fluid delivery device coupled to the fluid-conducting lumen.

22. The apparatus according to claim 21, wherein said controller further comprises feedback mechanism.

23. The apparatus according to claim 17, further comprising a temperature sensor located on said tip.

24. A method for tissue treatment, said method comprising:
introducing a needle into a target volume including at least a portion of adipose tissue, said needle including:
a light guide,
at least one RF electrode, and
at least one fluid conducting channel;
delivering RF energy to the at least one electrode thereby heating said target volume; and
operating one or more laser sources to deliver laser energy through said light guide to said target volume.

25. The method according to claim 24, wherein the RF energy and the laser energy are provided in at least partially overlapping periods of time.

26. The method according to claim 24, wherein at least one laser source is operated in a continuous operation mode and at least one laser source is operated in a pulse operation mode.

27. The method according to claim 24, further comprising providing a means for visual observation of the tip of said needle in said target volume.

28. The method according to claim 23, further comprising delivering or removing through the fluid conducting channel at least one fluid selected from a group of fluids consisting of a cooling fluid, heating fluid, conductivity changing fluid, or products of adipose tissue treatment.

29. A method of tissue treatment, said method comprising:
applying a first electrode to the outer surface of a subject's skin and introducing a needle subcutaneously to a target volume, said needle having a second electrode;
providing a radio frequency energy between said first electrode and said second electrode;
applying laser radiation to at least a volume of the tissue surrounding said second electrode, said radiation being conducted through said needle; and
changing the tissue state.

30. The method according to claim 29, wherein the change of the tissue state includes at least one of the effects from the group of effects including adipose tissue destruction, shrinking, breakdown, and skin tightening.

31. The method according to claim 29, wherein the radio frequency energy is applied within the range of 100 Khz to 100 Mhz.

32. The method according to claim 29, wherein said laser radiation is applied in a manner such at it at least partially overlaps in time with the provision of radio frequency.

33. The method according to claim 29, further comprising altering the volume at the treated volume of tissue at least one of a group of fluids consisting of a cooling fluid, heating fluid, conductivity changing fluid, or products of adipose tissue treatment.

34. A method of adipose tissue treatment, said method comprising:
applying at least two electrodes to the skin of a subject;
generating a radio frequency field between said electrodes;
introducing subcutaneously a light guide and locating said guide such that at least a section of the light guide is located in said radio frequency field; and
irradiating by laser radiation the part of said skin located in said radio frequency field.

35. The method according to claim 34, wherein said radio frequency and said laser radiation is provided in such a manner to effectuate the changing of the state of said skin.

36. The method according to claim 34, wherein said changing state includes at least one of a group consisting of adipose tissue destruction, shrinking, breakdown, and skin tightening.

37. A method of lipo-sculpturing a segment of subject's body, said method comprising:
providing at least two sources of electromagnetic energy located in distant regions of the electromagnetic energy spectrum;
delivering the energy generated by the first source by contact with the skin to a target volume of the tissue;
introducing subcutaneously said second electromagnetic energy source and locating it such that it delivers the energy generated by the second source to said target volume of the tissue;
coupling to said target volume energy emitted by both sources; and
changing the state of said target volume of the tissue.

38. The method of lipo-sculpturing a segment of human body according to claim 36, wherein said method further comprises contraction of at least collagen containing tissue.

39. A method of adipose tissue treatment, said method comprising:
applying electromagnetic radiation generated by two different electromagnetic radiation sources to a target volume of tissue, where the first source of electromagnetic radiation is applied externally such that said radiation penetrates the surface of said tissue and is concentrated in the target volume and the second source of electromagnetic radiation is applied to the same target volume by the second source located in said volume;
setting the energy level of the first source to a level insufficient to produce the desired treatment effect; and
setting the energy level of the second source to a level that when combined with the first source the combination is sufficient to produce a treatment effect.

40. The method according to claim 39, wherein said first source of energy is a source of radio frequency radiation.

41. The method according to claim 39, wherein said second source of energy is a source of infrared radiation.

42. The method according to claim 39, further comprising altering at said target volume the amount of fluid of at least one of a group of fluids consisting of a cooling fluid, a heating fluid, a conductivity changing fluid, or products of adipose tissue treatment.

43. A method for adipose tissue treatment, said method comprising:
introducing a needle into a target volume of adipose tissue, said needle comprising:
a light guide operatively configured to deliver laser radiation to the target volume;
at least one RF electrode operatively configured to deliver RF radiation to the target volume, and
at least one fluid conducting channel;
delivering at least one of the radiations to the target volume to destroy the adipose tissue at the target volume; and
operating a mechanism to remove from said target volume radiation adipose tissue interaction products.
